(19) United States
(12) Patent Application Publication
(10) Pub. No.: US 2017/0071967 A1
(43) Pub. Date: Mar. 16, 2017

(54) IMMUNOGENIC LIPOSOMAL FORMULATION

(71) Applicant: GlaxoSmithKline Biologicals, SA, Rixensart (BE)

(72) Inventors: Nupur DUTTA, Vernon Hills, IL (US); Hardeep OBEROI, Hamilton, MT (US); David BURKHART, Hamilton, MT (US); Jay T EVANS, Hamilton, MT (US)

(21) Appl. No.: 15/125,387

(22) PCT Filed: Mar. 12, 2015
(86) PCT No.: PCT/IB2015/051810

§ 371 Date: Sep. 12, 2016

Related U.S. Application Data
(60) Provisional application No. 61/952,118, filed on Mar. 12, 2014.

(51) Int. Cl. A61K 31/7028; A61K 47/22; A61K 9/127

(52) U.S. Cl. CPC A61K 31/7028; A61K 9/127; A61K 47/22; A61K 9/1272; A61K 9/1277

(57) ABSTRACT

A liposomal composition comprising a liposome and an aminoalkansulfonic buffer is described and claimed.

### STATEMENT REGARDING FEDERALLY SPONSORED RESEARCH

Aspects of this invention were made with United States government support pursuant to NIH Contract #HHSN272200900008C, the United States government may have certain rights in the invention

### BACKGROUND

Toll-like receptor 4 (TLR4) agonists are immunogenic compounds. TLR-4 agonists have been formulated in liposomes for delivery. Monophosphoryl Lipid A is a known TLR 4 agonist. 3-O-deacylated Monophosphoryl Lipid A (MPL) is formulated in liposomal compositions in vaccines. There is a need for improved liposomal compositions in general and in particular for improved liposomal compositions of TLR4 agonists for administration to a human subject. Liposomal compositions of potent TLR4 agonists having high incorporation efficiency are desirable.

### SUMMARY OF THE INVENTION

The present invention is directed to improved liposomes for use in pharmaceutical compositions.

In one embodiment, the present invention provides a liposomal composition comprising lipids, suitably phospholipids and an aminoalkansulfonic buffer such as HEPES, HEPPS/EPPS, MOPS, MOBS and PIPES.

In another suitable embodiment, the present invention provides a liposomal composition comprising lipids such as phospholipids, and an aminoalkyl glucosaminide phosphate (AGP), suitably CRX-601.

In another suitable embodiment, the present invention provides a liposomal composition comprising lipids, an AGP and an aminoalkansulfonic buffer wherein the lipids are suitably phospholipids.

In another embodiment, the present invention provides a process for improved production of a liposomal composition comprising the steps of: dissolving a lipid, such as dioleoyl phosphatidylcholine (generally, “DOPC”), (optionally with cholesterol and/or a pharmaceutically active ingredient, such as an AGP) in organic solvent, removing the solvent to yield a phospholipid film, adding the film to HEPES buffer, dispersing the film into the solution, and extruding the solution successively through polycarbonate filters to form unilamelar liposomes. The liposomal composition can additionally be aseptically filtered.

These novel liposomal compositions have remarkably high incorporation efficiency with AGPs, which are known to be potent and potentially reactogenic. Formulating a liposomal composition of AGP for pharmaceutical use as describe herein may result in an improved therapeutic index for the composition when compared to other formulations of the agonist.

In one suitable embodiment, a liposomal composition exhibits high incorporation of TLR4 agonists when the liposome is formed with cholesterol, but also when the liposome is formed without cholesterol, providing advantages for production and formulation of such liposomal compositions.

The liposomes of the present invention are beneficial in both the production and in the use of a pharmaceutical composition.

Additional embodiments are disclosed in the descriptions, figures and claims provided herein.

### BRIEF DESCRIPTION OF THE DRAWINGS

FIG. 1 LAL data showing incorporation efficiency as determined by comparing the slope and onset time of the sample with respect to the CRX-601 IN reference (0% incorporation).

FIG. 2 LAL data showing incorporation efficiency as determined by comparing the slope and onset time of the sample with respect to the CRX-601 IN reference (0% incorporation).

FIG. 3 LAL data showing incorporation efficiency as determined by comparing the slope and onset time of the sample with respect to the CRX-IN reference (0% incorporation).

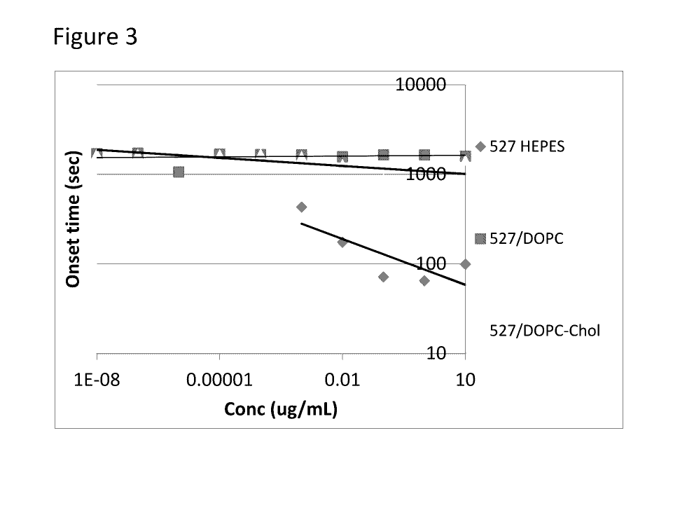

### DETAILED DESCRIPTION OF THE INVENTION

Liposomes

The term “liposome(s)” generally refers to uni- or multilamellar (particularly 2, 3, 4, 5, 6, 7, 8, 9, or 10 lamellar depending on the number of lipid membranes formed) lipid structures enclosing an aqueous interior. Liposomes and liposome formulations are well known in the art. Lipids which are capable of forming liposomes include all substances having fatty or fat-like properties. Lipids which can make up the lipids in the liposomes may be selected from the group comprising glycerides, glycerophospholipides, glycerophosphinolipids, glycerophosphonolipids, sulfolipids, sphingolipids, phospholipids, isoprenolides, steroids, stearines, sterols, archeolipids, synthetic cationic lipids and carbohydrate containing lipids.

In a particular embodiment of the invention the liposomes comprise a phospholipid. Suitable phospholipids include (but are not limited to): phosphocholine (PC) which is an intermediate in the synthesis of phosphatidylcholine; natural phospholipid derivates: egg phosphocholine, egg phosphocholine, soy phosphocholine, hydrogenated soy phosphocholine, sphingomyelin as natural phospholipids; and synthetic phospholipid derivates: phosphocholine (didecanoyl-L-α-phosphatidylcholine [DDPC], dilauroylphosphatidylcholine [DLPC], dimyristoylphosphatidylcholine [DMPC], dipalmitoyl phosphatidylcholine [DPPC], Distearoyl phosphatidylcholine [DSPC], Dioleoyl phosphatidylcholine [DOPC], 1-palmitoyl, 2-oleoylphosphatidylcholine [POPC], Dielaidoyl phosphatidylcholine [DEPC]), phosphoglycerol (1,2-Dimyristoyl-sn-glycero-3-phosphoglycerol [DMPG], 1,2-dipalmitoyl-sn-glycero-3-phosphoglycerol [DPPG], 1,2-distearoyl-sn-glycero-3-phosphoglycerol [DSPG], 1-palmitoyl-2-oleoyl-sn-glycero-3-phosphoglycerol [POPG]), phosphatidic acid (1,2-dimyristoyl-sn-glycero-3-phosphatidic acid [DMPA], dipalmitoyl phosphatidic acid [DPPA], distearoyl-phosphatidic acid [DSPA]), phosphoethanolamine (1,2-dimyristoyl-sn-glycero-3-phosphoethanolamine [DMPE], 1,2-Dipalmitoyl-sn-glycero-3-phosphoethanolamine [DPPE], 1,2-distearoyl-sn-glycero-3-phosphoethanolamine DSPE 1,2-Dioleoyl-sn-Glycero-3-Phosphoethanolamine [DOPE]), phoshoserine, polyethylene glycol [PEG] phospholipid (mPEG-phospholipid, polyglycerin-phospholipid, functionalized-phospholipid, terminal activated-phospholipids) 1,2-dioleoyl-3-(trimethylammonium) propane (DOTAP). In one embodiment the liposomes comprise 1-palmitoyl-2-oleoyl-glycero-3-phosphoethanolamine. In one embodiment highly purified phosphatidylcholine is used and can be selected from the group comprising Phosphatidylcholine (EGG), Phosphatidylcholine Hydrogenated (EGG), Phosphatidylcholine (SOY) and Phosphatidylcholine Hydrogenated (SOY). In a further embodiment the liposomes comprise phosphatidylethanolamine [POPE] or a derivative thereof or may comprise Sphingomylen (SPNG).

Liposome size may vary from 30 nm to several 5 μm depending on the phospholipid composition and the method used for their preparation. In particular embodiments of the invention, the liposome size will be in the range of 30 nm to 500 nm and in further embodiments 50 nm to 200 nm, suitably less than 200 nm. Dynamic laser light scattering is a method used to measure the size of liposomes well known to those skilled in the art.

In particular liposomes of the invention may comprise dioleoyl phosphatidylcholine [DOPC] and a sterol, in particular cholesterol.

Liposomal Composition

A “liposomal composition” is a prepared composition comprising a liposome and the contents within the liposome, particularly including the lipids which form the liposome bilayer(s), compounds other than the lipids within the bi-layer(s) of the liposome, compounds within and associated with the aqueous interior(s) of the liposome, and compounds bound to or associated with the outer layer of the liposome. Thus, in addition to the lipids of the liposome, a liposomal composition of the present invention suitably may include, but is not limited to, pharmaceutically active ingredients, vaccine antigens and adjuvants, excipients, carriers and buffering agents. In a preferred embodiment, such compounds are complementary to and/or are not significantly detrimental to the stability or AGP-incorporation efficiency of the liposomal composition.

“Liposomal formulation” means a liposomal composition, such as those described herein, formulated suitably with other compounds for storage and/or administration to a subject.

Thus, a liposomal formulation of the present invention, includes a liposomal composition of the present invention, and may additionally include, but is not limited to, liposomal compositions outside the scope of the present invention, as well as pharmaceutically active ingredients, vaccine antigens and adjuvants, excipients, carriers and buffering agents. In a preferred embodiment, such compounds are complementary to and/or are not significantly detrimental to the stability or AGP-incorporation efficiency of the liposomal composition of the present invention.

Aminoalkyl Glucosaminide Phosphate Compounds. AGPs are Toll-Like Receptor 4 (TLR4) modulators. Toll-like receptor 4 recognizes bacterial LPS (lipopolysaccharide) and when activated initiates an innate immune response. AGPs are a monosaccharide mimetic of the lipid A protein of bacterial LPS and have been developed with ether and ester linkages on the “acyl chains” of the compound. Processes for making these compounds are known and disclosed, for example, in WO 2006/016997, U.S. Pat. Nos. 7,288,640 and 6,113,918, and WO 01/90129, which are hereby incorporated by reference in their entireties. Other AGPs and related processes are disclosed in U.S. Pat. No. 7,129,219, U.S. Pat. No. 6,525,028 and U.S. Pat. No. 6,911,434. AGPs with ether linkages on the acyl chains employed in the composition of the invention are known and disclosed in WO 2006/016997 which is hereby incorporated by reference in its entirety. Of particular interest, are the aminoalkyl glucosaminide phosphate compounds set forth and described according to Formula (III) at paragraphs [0019] through [0021] in WO 2006/016997.

Aminoalkyl glucosaminide phosphate compounds employed in the present invention have the structure set forth in Formula 1 as follows:

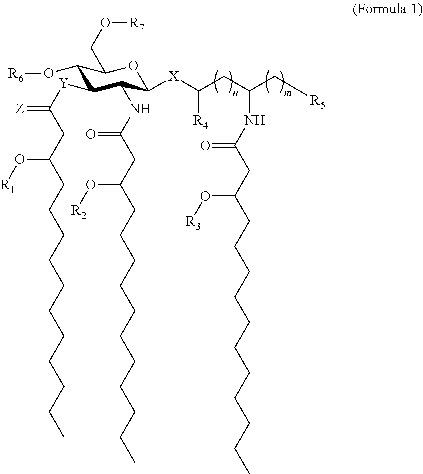

wherein
m is 0 to 6
n is 0 to 4;
X is O or S, preferably O;
Y is O or NH;
Z is O or H;
each R₁, R₂, R₃ is selected independently from the group consisting of a C₁₋₂₀ acyl and a C₁₋₂₀ alkyl;
R₄ is H or Me;
R₅ is selected independently from the group consisting of —H, —OH, —(C₁-C₄) alkoxy, —PO₃R₈R₉, —OPO₃R₈R₉, —SO₃R₈, —OSO₃R₈, —NR₈R₉, —SR₈, —CN, —NO₂, —CHO, —CO₂R₈, and —CONR₈R₉, wherein R₈ and R₉ are each independently selected from H and (C₁-C₄) alkyl; and
each R₆ and R₇ is independently H or PO₃H₂.

In Formula 1 the configuration of the 3′ stereogenic centers to which the normal fatty acyl residues (that is, the secondary acyloxy or alkoxy residues, e.g., R₁O, R₂O, and R₃O) are attached is R or S, preferably R (as designated by Cahn-Ingold-Prelog priority rules). Configuration of aglycon stereogenic centers to which R₄ and R₅ are attached can be R or S. All stereoisomers, both enantiomers and diastereomers, and mixtures thereof, are considered to fall within the scope of the present invention.

The number of carbon atoms between heteroatom X and the aglycon nitrogen atom is determined by the variable “n”, which can be an integer from 0 to 4, preferably an integer from 0 to 2.

The chain length of normal fatty acids R₁, R₂, and R₃ can be from about 6 to about 16 carbons, preferably from about 9 to about 14 carbons. The chain lengths can be the same or different. Some preferred embodiments include chain lengths where R1, R2 and R3 are 6 or 10 or 12 or 14.

Formula 1 encompasses L/D-seryl, -threonyl, -cysteinyl ether and ester lipid AGPs, both agonists and antagonists and their homologs (n=1-4), as well as various carboxylic acid bioisosteres (i.e, R₅ is an acidic group capable of salt formation; the phosphate can be either on 4- or 6-position of the glucosamine unit, but preferably is in the 4-position).

In a preferred embodiment of the invention employing an AGP compound of Formula 1, n is 0, R₅ is CO₂H, R₆ is PO₃H₂, and R₇ is H. This preferred AGP compound is set forth as the structure in Formula 1a as follows:

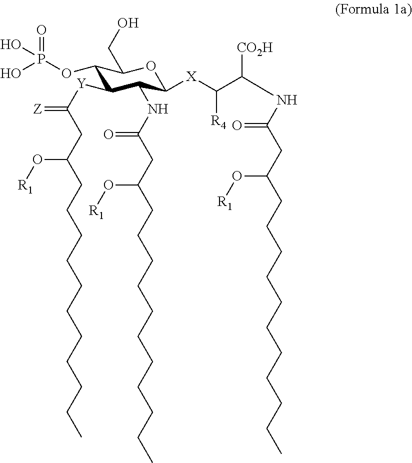

wherein X is O or S; Y is O or NH; Z is O or H; each R₁, R₂, R₃ is selected independently from the group consisting of a C₁₋₂₀ acyl and a C₁₋₂₀ alkyl; and R₄ is H or methyl.

In Formula 1a the configuration of the 3′ stereogenic centers to which the normal fatty acyl residues (that is, the secondary acyloxy or alkoxy residues, e.g., R₁O, R₂O, and R₃O) are attached as R or S, preferably R (as designated by Cahn-Ingold-Prelog priority rules). Configuration of aglycon stereogenic centers to which R₄ and CO₂H are attached can be R or S. All stereoisomers, both enantiomers and diastereomers, and mixtures thereof, are considered to fall within the scope of the present invention.

Formula 1a encompasses L/D-seryl, -threonyl, -cysteinyl ether or ester lipid AGPs, both agonists and antagonists.

In both Formula 1 and Formula 1a, Z is O attached by a double bond or two hydrogen atoms which are each attached by a single bond. That is, the compound is ester-linked when Z═Y═O; amide-linked when Z═O and Y═NH; and ether-linked when Z═H/H and Y═O.

Especially preferred compounds of Formula 1 are referred to as CRX-601 and CRX-527. Their structures are set forth as follows:

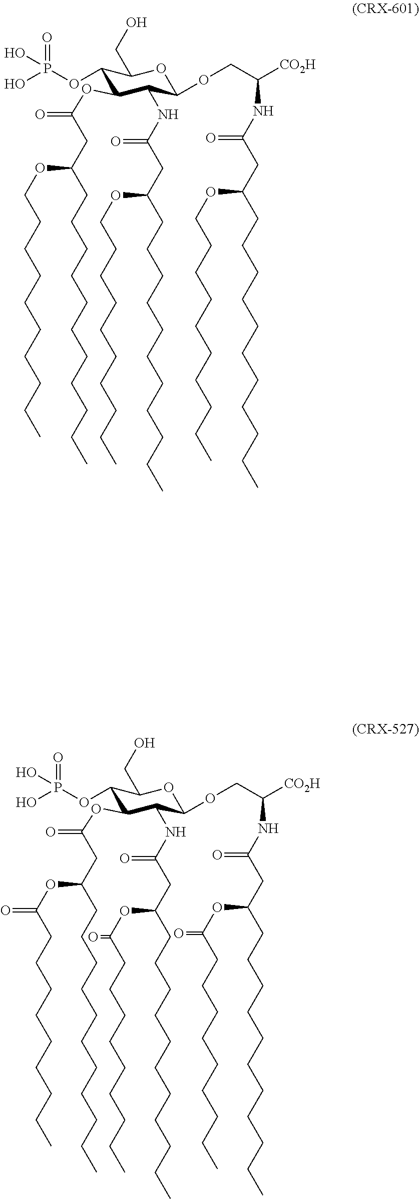

Additionally, another preferred embodiment employs CRX 547 having the structure shown.

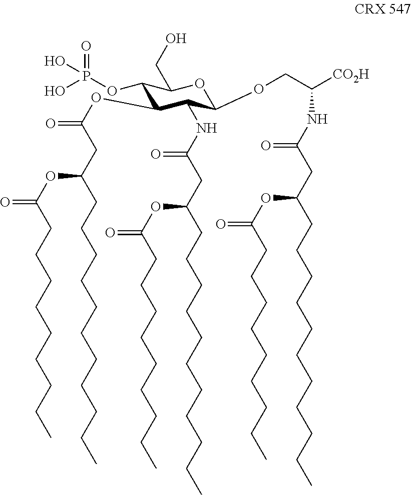

Still other embodiments include AGPs such as CRX 602 or CRX 526 providing increased stability to AGPs having shorter secondary acyl or alkyl chains.

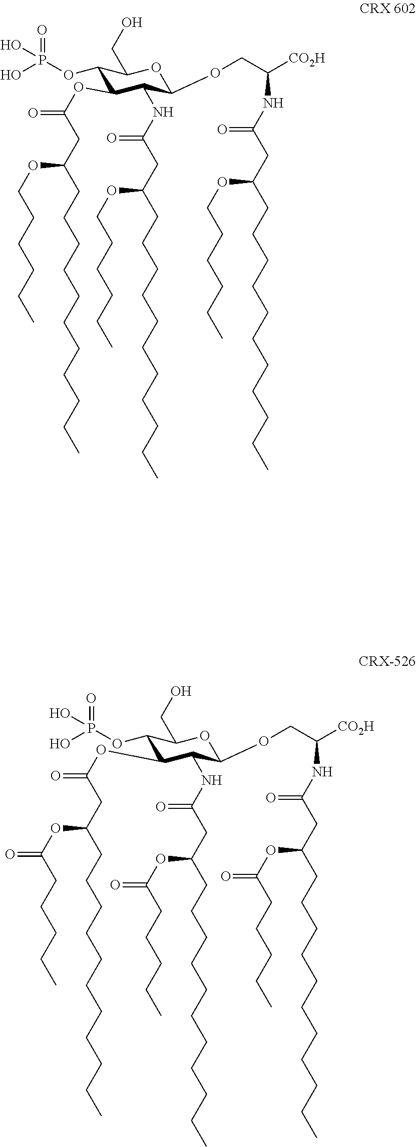

Other AGPs suitable for use in the present invention include CRX 524 and CRX 529.

Buffers.

In one embodiment of the present invention, a liposomal composition is buffered using a zwitterionic buffer. Suitably, the zwitterionic buffer is an aminoalkanesulfonic acid or suitable salt. Examples of aminoalkanesulfonic buffers include but are not limited to HEPES, HEPPS/EPPS, MOPS, MOBS and PIPES. Preferably, the buffer is a pharmaceutically acceptable buffer, suitable for use in humans, such as in for use in a commercial injection product. Most preferably the buffer is HEPES. The liposomal composition may suitable include an AGP.

In suitable embodiments of the present invention the liposomes are buffered using HEPES having a pH of about 7.

In a preferred embodiment of the present invention the AGPs CRX-601, CRX-527 and CRX-547 are included in a liposomal composition buffered using HEPES having a pH of about 7. The buffers may be used with an appropriate amount of saline or other excipient to achieve desired isotonicity. In one preferred embodiment 0.9% saline is used.

HEPES: CAS Registry Number: 7365-45-9 C₈H₁₈N₂O₄S

1-Piperazineethanesulfonic acid, 4-(2-hydroxyethyl)

HEPES is a zwitterionic buffer designed to buffer in the physiological pH range of about 6 to about 8 (e.g. 6.15-8.35) and more specifically from a more useful range of about 6.8 to about 8.2 and, as in the present invention, between about 7 and about 8 or between 7 and 8, and preferably between about 7 and less than 8. HEPES is typically a white crystalline powder and has the molecular formula: C₈H₁₈N₂O₄S of the following structure:

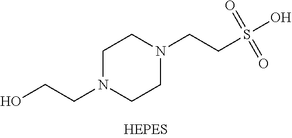 [SMILES: O=S(=O)(O)CCN1CCN(CCO)CC1]

HEPES is well-known and commercially available. (See, for example, Good et al., Biochemistry 1966.)

Liposome Preparation

Standard methods for making liposomes include, but are not limited to methods reported in Liposomes: A Practical Approach, V. P. Torchilin, Volkmar Weissig Oxford University Press, 2003 and are well known in the art.

In one suitable process for making a liposomal composition of the present invention an AGP (e.g. CRX-601 (20 mg)) and DOPC (specifically, 1,2-Dioleoyl-sn-glycero-3-phosphocholine) (400 mg)) and optionally a sterol (e.g. cholesterol (100 mg)) are dissolved as in an organic phase of chloroform or tetrahydrofuran in a round bottom flask. The organic solvent is removed by evaporation on a rotary evaporator and further with high pressure vacuum for 12 hrs. To the mixed phospholipid film thus obtained is added 10 ml of an aminoalkanesulfonic buffer such as 10 mM HEPES or 10 mM HEPES-Saline buffer pH 7.2. The mixture is sonicated on a water bath (20-30° C.) with intermittent vortexing until all the film along the flask walls is dispersed into the solution (30 min-1.5 hrs). The solution is then extruded successively through polycarbonate filters with the aid of a lipid miniextruder (Lipex™ extruder (Northern Lipids Inc., Canada)) to form unilamellar liposomes. The liposome composition is then aseptically filtered using a 0.22 μm filter into a sterile depyrogenated container. The average particle size of the resultant formulation as measured by dynamic light scattering is 80-120 nm with a net negative zeta-potential. The formulation represents final target concentrations of 2 mg/mL CRX- 601, 10 mg/mL cholesterol, and 40 mg/mL total phospholipids.

The aminoalkyl glucosaminide 4-phosphate (AGP) CRX-601 used in this work can be synthesized as described previously {Bazin, 2008 32447/id}, and purified by chromatography (to >95% purity). CRX-601, either in the starting material or in the final product can be quantified by a standard reverse phase HPLC analytical method.

CRX-601 formulated in the HEPES buffer (pH=7.0) five times faster obtained the desire reduction of particle size five times faster, as compared to liposome hydration buffer (“LHB,” phosphate based, pH=6.1). The rehydration of the CRX-601 lipid films in the HEPES buffer required four times less total pressure and time to formulate the liposomes as compared to the LHB phosphate buffer. This is a significant improvement since it saves both energy and time and puts much less stress on the AGPs during the processing of the liposomes.

In one one embodiment suitable ranges of components of a liposomal composition comprise a lipid in a range of about 3-4% w/v, a sterol at 1% w/v, an active, such as an AGP, in range of 0.1-1% w/v and an aminoalkanesulfonic buffer at 10 mM. In one embodiment sterol is suitably present a range of 0.5-4% w/v. Additionaly in one embodiment the lipid:sterol:active ratio is about 3-4:1:0.1-1.

### EXAMPLES

### Example 1

CRX-601 Formulation Lipid Compatibility Study.

Eight lipids were screened in a study with CRX-601 to find the optimal liposome formulation for CRX-601 leading to maximum stability of the API (CRX-601) and acceptable pyrogenicity and/or toxicity that may be related to the adjuvant.

The Lipex Extruder™ was used to prepare the formulations. The 10 mM HEPES at pH=7.0 was selected as the hydration buffer. The liposomal formulations were prepared at a target concentration of 2 mg/mL in the HEPES buffer at pH=7.0. These lipid formulations were put on real time stability at 2-8° C. for 6 months and accelerated stability at 40° C. for 14 days to monitor the degradation of CRX-601 by RP-HPLC over time, along with any changes in appearance, particle size, and zeta potential to account for aggregation, and chromogenic limulus amebocyte lysate (LAL) to account for changes in the percent incorporation of CRX-601.

Table 1 shows the t=0 (process data) for all the liposomes prepared.

TABLE 1

| | Phospholipid | | | | | | Conc. |
|---|---|---|---|---|---|---|---|
| | combinations | | | | Avg. | | by |
| | with CRX-601 | Appearance | Processing | | particle | Zeta | RP- |
| | in HEPES at | of the | Sonication | # | size | Potential | HPLC |
| | pH = 7.0 | formulation | time (min) | passes | (nm) | (mV) | (mg/mL) |
| 1 | DOPC/CHOL | translucent | 30 | 1 | 120 | −19.6 | 2.0 |
| 2 | DOPC/DC- | translucent | 30 | 1 | 85 | 24.9 | 1.1 |
| | CHOL | | | | | | |
| 3 | DOPC | milky | 110 | 2 | 145 | −21.9 | 2.3 |
| 4 | DPPC/CHOL | translucent | 60 | 1 | 121 | −27.5 | 2.1 |
| 5 | DLPC/CHOL | translucent | 60 | 1 | 110 | −15.2 | 1.9 |
| 6 | E. coli | milky | 90 | 3 | 181 | −18.7 | 0.2 |
| | PE/CHOL | | | | | | |
| 7 | DSPC/CHOL | translucent | 70 | 1 | 78 | −14.3 | 2.2 |
| 8 | DSPC | translucent | 90 | 4 | 64 | −11.1 | 1.7 |
| 9 | PC/CHOL | translucent | 60 | 1 | 116 | −28.4 | 2.4 |
| 10 | SPNG/CHOL | milky | 100 | 4 | 184 | −33.2 | 2.1 |
| 11 | DOTAP/CHOL | translucent | 90 | 1 | 124 | 21.4 | 2.2 |

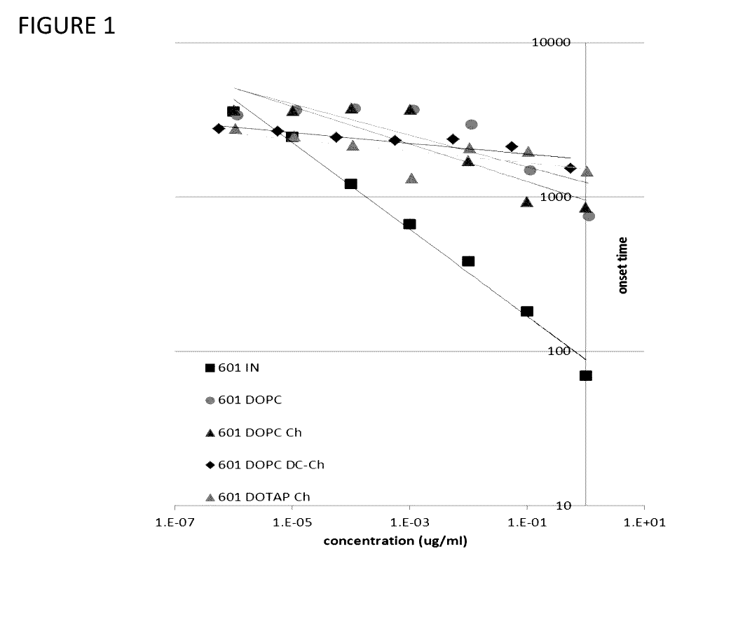

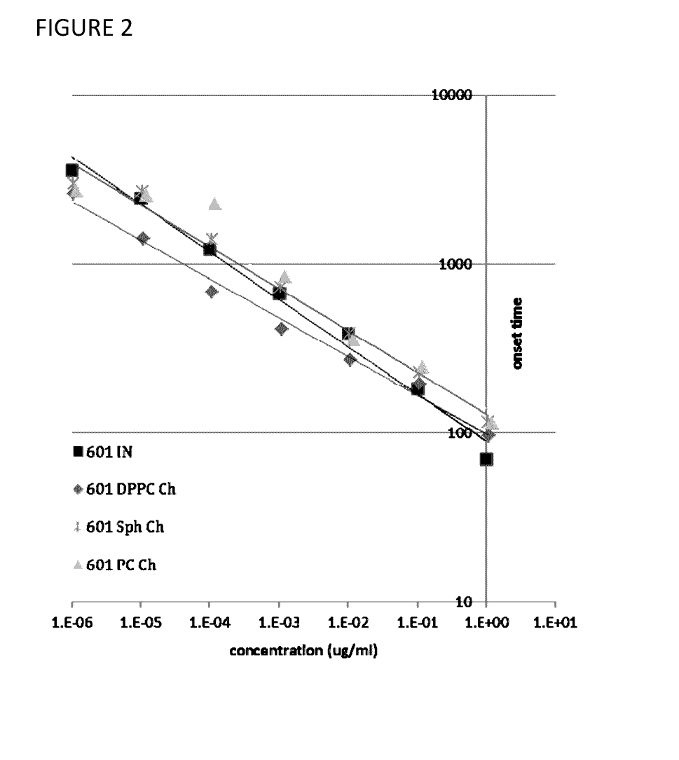

Incorporation efficiency was determined by comparing the slope and onset time of the sample with respect to the CRX-601 IN reference (0% incorporation) from the LAL data. LAL data at t=0 shown below showed good incorporation for CRX-601 in DOPC, DOPC Chol, DOPC DC-Chol, DOTAP, and DOTAP Chol. The rest of the formulations showed poor incorporation as seen in the following FIGS. 1 and 2.

### Example 2

### Incorporation Efficiency Testing

To determine the effect of cholesterol on incorporation of CRX-601 into the liposomes, LAL assays were performed on various liposomal compositions with and without cholesterol. The data obtained (not shown) confirm earlier LAL work showing that DOPC and DOPC-cholesterol compositions have surprisingly high levels of incorporation of CRX 601. However, the results of these LAL assays were not sufficiency sensitive or consistent to draw conclusions with respect to the effect of cholesterol on incorporation.

Rabbit pyrogenicity tests were used as a surrogate measure of CRX-601 incorporation into liposomes and as a measure of their stability in biological milieu. The tests were performed at Pacific Biolabs (Hercules, Calif.) as per their SOP 16E-02. The individual temperature increases from three rabbits per test are indicated in the table below.

The data from Table 2 indicate that the DOPC liposome formulations with up to 4 mg CRX-601/ml prepared with or without cholesterol are non-pyrogenic up to a dose of 1000 ng/kg. This lack of pyrogenicity corresponds to a 400 fold improvement over free CRX-601 (max non-pyrogenic dose of 2.5 ng/kg), and indicates a >99% incorporation of CRX-601 into the liposome bilayer.

TABLE 2
Representative rabbit pyrogen test measurements for DOPC liposome formulations prepared with
or without cholesterol. Values in parenthesis are maximum temperature change for three animals
during the testing period. A temperature rise of 0.5° C. or more is considered a pyrogenic
response. The symbols P and F indicate a ‘Pass’ or ‘Fail’ response respectively.

| | Max Temp. Rise observed | Max Temp. Rise observed |
|---|---|---|
| | at any time interval | at any time interval |
| | (compared to controls) | (compared to controls) |
| Formulation | at a dose of 500 ng/kg | at a dose of 1000 ng/kg |
| CRX-601 DOPC liposomes prepared at | P (0.3° C., 0.0° C., 0.0° C.) | P (0.0° C., 0.0° C., 0.0° C.) |
| a targeted concentration of 2 mg/mL | | |
| CRX-601 DOPC-Cholesterol liposomes | P (0.3° C., 0.0° C., 0.4° C.) | P (0.1° C., 0.4° C., 0.4° C.) |
| prepared at a targeted concentration | | |
| of 2 mg/mL | | |
| CRX-601 DOPC liposomes prepared at | P (0.1° C., 0.0° C., 0.2° C.) | P (0.0° C., 0.0° C., 0.4° C.) |
| a targeted concentration of 4 mg/mL | | |
| CRX-601 DOPC-Cholesterol liposomes | P (0.0° C., 0.0° C., 0.0° C.) | P (0.3° C., 0.4° C., 0.2° C.) |
| prepared at a targeted concentration | | |
| of 4 mg/mL | | |

The data from Table 3 indicate that the DOPC cholesterol liposome formulations with up to 8 mg CRX-601/ml are non-pyrogenic up to a dose of 500 ng/kg. This lack of pyrogenicity corresponds to a 200 fold improvement over free CRX-601 (max non-pyrogenic dose of 2.5 ng/kg), and indicates a >99% incorporation of CRX-601 into the liposome bilayer.

TABLE 3
Table 3: Representative rabbit pyrogen test measurements for DOPC liposome formulations prepared
with cholesterol. Values in parenthesis are maximum temperature change for three animals
during the testing period. A temperature rise of 0.5° C. or more is considered a pyrogenic
response. The symbols P and F indicate a ‘Pass’ or ‘Fail’ response respectively.

| | Max Temp. Rise observed | Max Temp. Rise observed |
|---|---|---|
| | at any time interval | at any time interval |
| | (compared to controls) | (compared to controls) |
| Formulation | at a dose of 500 ng/kg | at a dose of 1000 ng/kg |
| CRX-601 DOPC Cholesterol liposomes | P (0.1° C., 0.1° C., 0.3° C.) | P (0.4° C., 0.3° C., 03° C.) |
| prepared at a targeted concentration | | |
| of 8 mg/mL | | |
| CRX-601 DOPC-Cholesterol liposomes | P (0.2° C., 0.4° C., 0.3° C.) | F (0.6° C., 1.0° C., 0.4° C.) |
| prepared at a targeted concentration | | |
| of 8 mg/mL | | |

CRX-527 is the ester analog of CRX 601. The data from Table 4 indicate that the DOPC cholesterol liposome formulations with up to 2 mg CRX-527/ml are non-pyrogenic up to a dose of 500 ng/kg. This lack of pyrogenicity suggests a very high (potentially >99%) incorporation of CRX-601 into the liposome bilayer. Interestingly, unlike CRX-601, CRX-527 in DOPC liposomal formulation (i.e. in the absence of cholesterol) was shown to be pyrogenic at 500 ng/kg.

TABLE 4

| | Max Temp. Rise observed | Max Temp. Rise observed |
|---|---|---|
| | at any time interval | at any time interval |
| | (compared to controls) | (compared to controls) |
| Formulation | at a dose of 500 ng/kg | at a dose of 1000 ng/kg |
| 527 DOPC liposomes prepared at a | F (0.9 C., 1.2 C., 1.0 C.) | F (0.7 C., 1.0 C., 1.3 C.) |
| targeted concentration of 2 mg/mL | | |
| 527 DOPC CHOL liposomes prepared at | P (0.4, 0.3, 0.2 C.) | P (0.1, 0.0, 0.1 C.) |
| a targeted concentration of 2 mg/mL | | |

Good incorporation results are also shown in Table 5 for cationic DOTAP and DOTAP-Cholesterol liposomes with CRX-601.

TABLE 5

| | Max Temp. Rise observed | Max Temp. Rise observed |
|---|---|---|
| | at anytime interval | at any time interval |
| | (compared to controls) | (compared to controls) |
| Formulation | at a dose of 500 ng/kg | at a dose of 1000 ng/kg |
| 601 DOTAP liposomes prepared at a | P (0.3 C., 0.3 C., 0.1 C.) | F (0.6 C., 0.6 C., 0.7 C.) |
| targeted concentration of 2 mg/mL | | |
| 601 DOTAP CHOL liposomes prepared at | P (0.2 C., 0.4 C., 0.4 C.) | F (0.6 C., 0.5 C., 0.4 C.) |
| a targeted concentration of 2 mg/mL | | |

1. A liposomal composition comprising a liposome and an aminoalkansulfonic buffer.

2. The liposomal composition of claim 1 wherein the aminoalkanesulfonic buffer is selected from the group comprising HEPES, HEPPS/EPPS, MOPS, MOBS and PIPES.

3. The liposomal composition of claim 1 wherein the aminoalkansulfonic buffer is HEPES.

4. The liposomal composition of claim 1 wherein the lipid which makes up the liposomes is selected from the group consisting of: glycerides, glycerophospholipides, glycerophosphinolipids, glycerophosphonolipids, sulfolipids, sphingolipids, phospholipids, isoprenolides, steroids, stearines, sterols, archeolipids, synthetic cationic lipids and carbohydrate containing lipids.

5. The liposomal composition of claim 1 wherein the lipids in the liposomes are phospholipids.

6. The liposomal composition of claim 1 wherein the lipids in the liposome are Dioleoyl phosphatidylcholine (DOPC).

7. The liposomal composition of claim 1 wherein the lipids in the liposome are 1,2-dioleoyl-3-(trimethylammonium) propane (DOTAP).

8. A liposome composition of claim 1 further comprising a sterol and in particular cholesterol.

9. A liposome composition comprising an AGP incorporated into liposome, wherein said liposome comprises dioleoyl phosphatidylcholine [DOPC] in the absence of a sterol.

10. A liposome composition of claim 9 wherein the AGP is CRX-601.

11. A liposome composition of claim 9 wherein the AGP is present in an amount greater than 10 mg/mL.

12. A liposome composition of claim 9 wherein the AGP is present in an amount less than 10, less than 9, less than 8, less than 7, less than 6, less than 5, less than 4, less than 3, less than 2 or less than 1 mg, but greater than 0 mg/mL.

13. A liposome composition of claim 9 wherein the AGP is present in an amount greater than 0 mg/mL but equal to or less than 10 mg/mL.

14. A liposome composition of claim 9 wherein the AGP is present in an amount between 30 μg/mL and 6 mg/mL.

15. The liposomal composition of claim 9 wherein the liposome is multilamellar

16. The liposomal composition of claim 9 wherein the liposome is 2, 3, 4, 5, 6, 7, 8, 9, or 10 lamellar.

17. The liposomal composition of claim 9 wherein the liposome is unilamellar.

18. The liposomal composition of claim 9 wherein the liposome size will be in the range of 50 nm to 500 nm and in further embodiments 50 nm to 200 nm.

19. The liposomal composition of claim 9 wherein the liposome size will be in the range of about 80-120 nm.

20. The liposomal composition of claim 9 wherein the liposomal structures encloses an aqueous interior.

21. The liposomal composition of claim 9 further comprising a lipid A mimetic, TLR4 ligand, or AGP.

22. The liposomal composition of claim 21 wherein the AGP is an aminoalkyl glucosaminide phosphate having the structure set forth in Formula I: 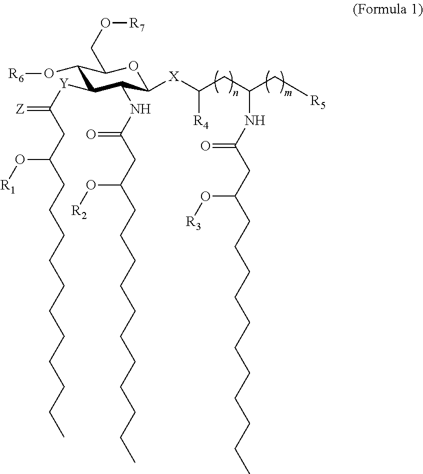
b. wherein
c. m is 0 to 6
d. n is 0 to 4;
e. X is O or S, preferably O;
f. Y is O or NH;
g. Z is O or H;
h. each R₁, R₂, R₃ is selected independently from the group consisting of a C₁₋₂₀ acyl and a C₁₋₂₀ alkyl;
i. R₄ is H or Me;
j. R₅ is selected independently from the group consisting of —H, —OH, —(C₁-C₄) alkoxy, —PO₃R₈R₉, —OPO₃R₈R₉, —SO₃R₈, —OSO₃R₈, —NR₈R₉, —SR₈, —CN, —NO₂, —CHO, —CO₂R₈, and —CONR₈R₉, wherein R₈ and R₉ are each independently selected from H and (C₁-C₄) alkyl; and
k. each R₆ and R₇ is independently H or PO₃H₂.

23. The liposomal composition of claim 1 further comprising an AGP selected from the group of CRX 527, 601, 602, 547, 529 and 529.

24. (canceled)

25. A method of improving the production of a liposomal composition comprising preparing a phospholipid film and adding to the phospholipd film to an aminoalkanesulfonic buffer

26. (canceled)

27. (canceled)

28. A process for production of a liposomal composition comprising the steps of:
a. dissolving a lipid, such as DOPC (optionally with cholesterol and/or a pharmaceutically active ingredient, such as an AGP) in organic solvent,
b. removing the solvent to yield a phospholipid film,
c. adding the phospholipid film to an aminoalkanesulfonic buffer,
d. dispersing the film into the solution, and
e. extruding the solution successively through polycarbonate filters to form unilamellar liposomes.

29. The process of claim 28 further comprising the step of aseptically filtering the extruded liposomes.
